(19) United States
(12) Patent Application Publication
(10) Pub. No.: US 2016/0000879 A1
(43) Pub. Date: Jan. 7, 2016

(54) Treatment of Smell and Taste Disorders Using Orally Administered Interferon-Alpha

(71) Applicant: Joseph Cummins, Edmond, OK (US)

(72) Inventor: Joseph Cummins, Edmond, OK (US)

(21) Appl. No.: 14/791,891

(22) Filed: Jul. 6, 2015

Related U.S. Application Data
(60) Provisional application No. 62/021,005, filed on Jul. 3, 2014.

(51) Int. Cl. A61K 38/21; A61K 9/00

(52) U.S. Cl. CPC A61K 38/212; A61K 9/0053; A61K 9/006; A61K 9/008

(57) ABSTRACT

The present disclosure is directed to the use of interferon to treat smell and taste disorders. In one embodiment a composition comprising interferon-alpha is administered orally to treat a patient suffering from diminished or distorted senses of smell and taste due to aging, medication usage, viral illness, physical trauma or a chronic medical condition.

### CROSS-REFERENCE TO RELATED APPLICATIONS

This application claims the benefit of U.S. Patent Application No. 62/021,005, a provisional utility application filed on Jul. 3, 2014. This application is made on the first USPTO business day following the anniversary of that earlier application and is therefore timely made.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

Smell and taste are a part of our sensing system much like vision and hearing. Molecules released by substances around us stimulate special nerve cells in the nose, mouth and throat. These special nerve cells transmit electric impulses to specific areas of the brain that recognize smell and taste.

Olfactory nerve cells, about six million per human nostril, are stimulated by odors around us such as flowers, baked goods, perfumes, etc. These olfactory nerve cells are located in a tiny patch of the mucous membrane high up in inner lining of the nose called the “smell organ.” Once stimulated, the smell organ sends its signals to the olfactory nerve, which continues on to the olfactory bulb. From there, the signal travels along the underside of the brain via the olfactory tract, which branches out to send signals to many parts of the brain, including areas involved in memory and emotion.

Taste receptor cells located in the taste buds of the mouth and throat react to food and drink, mixed with saliva. The human taste system consists of about 10,000 taste buds, each containing 100-150 taste cells. Taste buds are concentrated on the surface of the tongue, but also exist on the roof of the mouth, the back of the throat and on the larynx. Each taste receptor cell contains a nerve process that sticks out on the surface of the taste bud. The multiple nerve processes in each taste bud, the “microvilli,” send taste signals along nerve pathways to the brain.

Humans can commonly identify five basic taste sensations: sweet, sour, bitter, salty and umami (savory). However, an intact sense of smell is necessary to identify flavors such as chocolate. Saliva interacts with chocolate, releasing odor molecules that travel up the nasal passage from the back of the throat. The nasal cells are stimulated and the smell and flavor of chocolate will be recognized. Nine out of 10 people who complain of taste problems actually have a smell disorder that interferes with the ability to identify the flavor of foods.

The sensory system is also involved in the sense of taste. The fifth cranial (trigeminal) nerve carries pain, touch and temperature sensations to the brain from the face, eyes, mouth and tongue. The sensory system provides information about the temperature, texture and spiciness of food, but intact taste and smell systems are required to fully appreciate the flavor of food.

Over two million Americans complain of smell and taste problems, and over 250 thousand people in the US visit a physician for this problem each year. Twenty-five percent of the population over age fifty-five have an age-associated smell and taste impairment, and are often not aware of this problem. In the elderly, these disorders can lead to unexplained weight loss, depression and poor appetite.

Smell and taste disorders can be total (all odors or tastes), partial (affecting several odors or tastes), or specific (only one or a select few odors or tastes). Definitions of the various possible smell and taste disorders are as follows: Anosmia—Inability to detect odors; Hyposmia—Decreased ability to detect odors; Dysosmia—Distorted identification of smell; Parosmia—Altered perception of smell in the presence of an odor, usually unpleasant; Phantosmia—Perception of smell without an odor present; Agnosia—Inability to classify or contrast odors, although able to detect odors; Ageusia—Inability to taste; Hypogeusia—Decreased ability to taste; and Dysgeusia—Distorted ability to taste.

Smell and taste disorders don't simply mean a diminished quality of life. People with a faulty sense of smell and taste can be deprived of an important early warning system. Poisonous fumes, leaking gas, and spoiled foods can cause serious health problems if not recognized. Often people with smell and taste disorders increase their salt and sugar intake to improve the palatability of food, which can aggravate high blood pressure and diabetes.

The most common cause of smell and taste disorders is aging. Smell loss is more common than taste loss and occurs earlier in men. The sense of smell is most accurate between the ages of thirty and sixty. After age sixty, smell loss begins to accelerate, and by ages sixty-five to eighty, half of all people have moderate to severe loss. Seventy-five percent of the population above age eighty has moderate to severe smell loss.

Besides aging, about twenty percent of smell and taste disorders are due to viral infections, which damage the smell receptors in the nose. Another twenty percent are due to diseases of the nasal cavity, including polyps, sinusitis and allergies. An additional twenty percent are due to various medications, smoking, vitamin deficiency, brain tumors, chemical exposure, and the effects of radiation. Head trauma causes ten to twenty percent of smell and taste complaints. Many neurological disorders, like Alzheimer's disease, stroke, Parkinson's disease, and Lewy Body dementia are associated with smell and taste disorders. Other chronic conditions such as liver and kidney disorders, and gastroesophageal reflux disease (GERD) are often associated with taste disorders though most such patients have an intact sense of smell. As many as ten to twenty percent of taste and smell disorders do not have an identifiable cause.

There is currently no specific medication proven to be effective in treating smell and taste loss. Vitamin deficiencies can be corrected via supplementation. Stopping smoking may improve function, though it can take a number of years to restore lost sense of taste and smell. Medication contributing to smell and taste loss may be withdrawn or altered in dosage, but this is not always possible with critical medications. Taste and smell loss due to a viral infection can improve over time. The chance of recovery and amount of improvement is usually directly related to the severity of the infection and the amount of function initially lost. Taste and smell disorders due to MS and stroke usually improve with successful treatment of the underlying disease. However, this is not the case for other neurological conditions such as Alzheimer's and Parkinson's disease. For other chronic medical conditions, successful treatment of the underlying disease often results in improvement of taste and smell. Damage to the taste and smell centers caused by head injury generally is permanent.

“Interferon” is a term generically describing a distinct group of cytokines exhibiting pleiotropic activity generally categorized as antiviral, antiproliferative and immunomodulatory. In the early years of interferon research, an international committee was assembled to devise a system for orderly nomenclature of interferons and defined “interferon” as follows:

“To qualify as an interferon a factor must be a protein which exerts virus nonspecific, antiviral activity at least in homologous cells through cellular metabolic process involving synthesis of both RNA and protein.” Journal of Interferon Research, 1, pp. vi (1980).

“Interferon” as used herein in describing the present invention shall be deemed to have that definition and shall contemplate such proteins and glycoproteins, including for example, the subtypes interferon-alpha, interferon-beta, interferon-delta, interferon-epsilon, interferon-gamma, interferon-kappa, interferon-lambda, interferon-omega and interferon-tau, regardless of their source or method of preparation or isolation.

Originally identified for their ability to induce cellular resistance to viral infection, interferons are currently known to be potent mediators in the host defense mechanism and homeostasis, modulating both the innate and adaptive immune responses. Interferons are small, inducible, 20-25 KD, usually glycosylated proteins that are produced by vertebrate cells in response to various biological stimuli. Mechanistically, interferons mediate their biological activities by binding to receptors present on the surface of target cells. Specific ligand-receptor interactions trigger intracellular signaling cascade downstream, resulting in the synthesis of proteins that mediate mentioned pleiotropic activities.

Interferons are classified into two groups: type I or type II, based on their structure, physicochemical properties and biological activities. Type I and type II interferons exert their biological effects through different cellular receptors. In mammals, eight families of type I interferon have been described. These are: interferon-alpha, interferon-beta, interferondelta, interferon-epsilon, interferon-kappa, interferon-lambda, interferon-omega and interferontau. Among these families, interferon trophoblast, found only in ruminant ungulates, is not inducible by virus and is produced in the embryonic trophoectoderm at a specific time, early during pregnancy. Its major function is to create conditions for the completion of pregnancy. Interferon-delta, a polypeptide of about 149 amino acids, has been described only in pigs. This interferon is physiologically expressed by trophoblast during the period of implantation in uterus. Interferon-gamma is the sole representative of type II interferon in mammals.

As described herein a method for alleviating smell and taste disorders is provided. The method comprises administering interferon for contact with the mucosal membranes of the digestive and/or respiratory tracts.

### BRIEF SUMMARY OF THE INVENTION

A method of treating smell and taste disorders in an individual is provided. More particularly, a method for improving the sense of taste and/or smell in an individual suffering from partial to total loss or distortion of these senses is provided by administering low dose interferon to the individual. In one embodiment the method comprises treating smell and taste disorders associated with normal aging, medication usage, a past viral illness or injury, or an ongoing neurological or other chronic medical condition. In one embodiment the interferon is either interferon-alpha or interferon-beta and the interferon is administered orally, intranasally or by inhalation. The interferon is administered in an amount effective to improve the sense of taste and/or smell in an affected individual. In one embodiment the interferon is interferon-alpha and the interferon is administered orally, intranasally or by inhalation in an amount from about 0.1 IU/lb to about 100 IU/lb of patient body weight. When the interferon is administered by inhalation, a metered dose inhaler can be used that provides a dose from about 75 IU to about 1000 IU of interferon.

As disclosed herein a method is provided for treating disorders of the senses of taste and smell related to aging, medication usage, viral infection, physical trauma, and/or chronic disease. The method comprises orally administering interferon-alpha in an amount effective to restore some or all of the lost or distorted function of these senses.

### BRIEF DESCRIPTION OF THE SEVERAL VIEWS OF THE DRAWING

Not applicable.

### DETAILED DESCRIPTION OF THE INVENTION

Described herein is a Treatment of Smell and Taste Disorders Using Orally Administered Interferon-Alpha. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

In describing and claiming the invention, the following terminology will be used in accordance with the definitions set forth below.

As used herein, the term “treating” includes prophylaxis of the specific disorder or condition, or alleviation of the symptoms associated with a specific disorder or condition and/or preventing or eliminating said symptoms.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents and includes agents approved by a regulatory agency of the US Federal government or listed in the US Pharmacopeia for use in animals, including humans. The term “carrier” refers to a diluent, adjuvant, excipient or vehicle with which an active agent is administered.

A method is provided herein for treating taste and smell disorders related to aging, medication usage, viral infection, physical trauma, and/or chronic disease. The method comprises administering a low dose of interferon to a patient in need thereof. In one embodiment the interferon is interferon-alpha or interferon-beta, and more particularly, in one embodiment the administered biologically active interferon is human interferon-alpha.

The interferon can be administered to the patient through a number of routes, such as orally, intranasally, or by inhalation. The interferon containing composition can be administered in a single dose, or in several doses per day. In accordance with one embodiment the interferon is administered in a form of orally dissolving lozenges.

Interferon of human and murine origin is quantified in the art in terms of International Units (IU). Interferons of other than human or murine origin can be used in accordance with this invention. In that presently accepted practices may not extend the use of “International Units” to quantify non-human and non-murine interferons, it shall be understood that administration of amounts of non-human/non-murine interferons having the same efficacy as the quantities (IU's) of human interferon specified in this description are within the scope of the present invention.

As disclosed herein, a method for treating a patient suffering from disorders of the sense of taste and/or smell is provided wherein a composition comprising interferon is administered to the patient. In one embodiment the interferon is a type I interferon that is administered orally in a low dosage form and in one embodiment the interferon is interferonalpha. In one embodiment, wherein the interferon containing composition is administered orally, the composition is administered in a form or manner that optimizes contact of the composition with the oral and oral pharyngeal mucosa. In one embodiment the interferon containing composition is prepared as a lozenge, powder, liquid or chewable composition.

For the purpose of the present invention, interferon-alpha treatment dosages range from about 0.1 IU/lb to about 100 IU/lb of patient body weight, more typically about 0.5 to about 10 IU/lb of patient body weight. Thus, unit dosage forms for human use typically comprise about 5 IU to about 2500 IU of interferon-alpha, more typically about 10 IU to about 500 IU of interferon-alpha, in combination with a pharmaceutically acceptable carrier therefore. Dosage forms for treatment in accordance with this invention can be in solid, liquid, aerosol, ointment or cream formulation and are typically administered from one to four times daily until the condition being treated is alleviated. In one embodiment, human interferon-alpha is orally administered in a sterile aqueous solution. Chronic administration may be required for sustained benefit. Generally speaking, the dosage forms are administered in a disease state-dependent manner, including particularly administration bucally/sublingually, by oral ingestion or by inhalation.

As described in the following examples, low-dose human interferon-alpha (about 100-150 IU taken once daily) was administered in a liquid formulation to an adult male suffering from a complete loss of the sense of smell and to an adult female reporting a nearly complete loss of both smell and taste. Both subjects reported restoration of function within 4-6 weeks and subsequent loss of function after treatment was discontinued. The female subject received a second treatment course and again reported restoration of her ability to taste and smell. This second treatment course was with a liquid formulation of low-dose bovine interferon-alpha. Neither subject given low-dose oral formulations of interferon-alpha for treatment of smell and/or taste disorders reported any deleterious side effects.

### EXAMPLE 1

An adult female in her 50s suffering from systemic lupus erythematosus (SLE) and a nearly complete loss of smell and taste took low-dose human interferon-alpha liquid twice daily by mouth for 6 weeks at a dose of 100-150 IU. Following treatment, the subject reported that the taste of food had improved greatly. After discontinuing low-dose oral IFN treatment, the subject again gradually lost her sense of smell and taste. Upon realizing that she had now completely lost her sense of smell after being off oral interferon for several years, treatment was re-started with bovine interferon-alpha in a liquid preparation at a dose of 100-150 IU taken twice daily. By the second dose, the subject reported some improvement in her sense of taste. With continued treatment, the subject reported 50% improvement in smell and 75% improvement in her sense of taste. The subject reports that these improvements are maintained as long as she continues daily low-dose oral IFN treatment. Reversible loss of function is noted if treatment is discontinued for even a short length of time.

### EXAMPLE 2

A 69-year-old male who had completely lost his sense of smell at age 45 following 5 surgeries to remove nasal polyps took low-dose human interferon-alpha liquid once daily by mouth for 30 days at a dose of 100-150 IU. After treatment, the subject regained enough of his sense of smell to detect manure odor borne on the wind from cattle feedyards located 10-12 miles from his home. After discontinuing low-dose oral IFN treatment, the subject again completely lost his sense of smell.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A method for treating a patient suffering from smell and/or taste disorders is provided wherein a composition comprising interferon is administered to the patient.

2. The method of claim 1 wherein the interferon is a type I interferon.

3. The method of claim 1 wherein the interferon is administered orally.

4. The method of claim 1 wherein the interferon is administered bucally.

5. The method of claim 1 wherein the interferon is administered topically to the lung cells by inhalation.

6. The method of claim 5 wherein the interferon is interferon-alpha that is administered using a metered dose inhaler.

7. The method of claim 6 wherein a metered dose from the metered dose inhaler is from about 75 IU to about 1000 IU of interferon.

8. The method of claim 3 wherein the interferon is interferon-alpha and the effective amount administered is from about 0.1 IU/lb to about 100 IU/lb of patient body weight.

9. The method of claim 1 wherein the interferon is administered in an amount effective to improve a diminished or distorted sense of smell and/or taste that is due to aging, medication usage, viral infection, physical trauma, and/or chronic disease.

10. The method of claim 9 wherein the interferon is human, bovine or caprine interferon administered orally in a sterile aqueous solution.
